(19) United States
(12) Patent Application Publication
(10) Pub. No.: US 2015/0297171 A1
(43) Pub. Date: Oct. 22, 2015

(54) SYSTEMS AND METHODS FOR ACOUSTICALLY OR ELECTRONICALLY MONITORING CHEST SOUNDS

(71) Applicant: HD Medical Inc., Santa Clara, CA (US)

(72) Inventor: Arvind Thiagarajan, San Jose, CA (US)

(21) Appl. No.: 14/691,541

(22) Filed: Apr. 20, 2015

Related U.S. Application Data
(60) Provisional application No. 61/982,753, filed on Apr. 22, 2014.

(51) Int. Cl. A61B 7/04; A61B 5/00; A61B 7/00; H04R 5/027; A61B 5/0205

(52) U.S. Cl. CPC A61B 7/04; H04R 5/027; A61B 5/0205; A61B 7/003; A61B 5/0004; A61B 5/486; A61B 5/725

(57) ABSTRACT

The present invention relates to hybrid acoustic and electronic stethoscope. In one embodiment, the hybrid acoustic and electronic stethoscope includes a chest piece having a diaphragm, an acoustic selector, a microphone and a speaker. The microphone converts acoustic pressure signals from the diaphragm into corresponding electronic signals which can be outputted as processed acoustic signals via the speaker. The acoustic selector can select either the diaphragm or the speaker. A pair of binaurals is acoustically coupled to the acoustic selector via an acoustic conduit. In turn, the binaurals provides the acoustic pressure signals or the processed acoustic signals to a user.

### CROSS REFERENCE TO RELATED APPLICATIONS

This application claims the benefit of and is a continuation-in-part of co-pending U.S. provisional application No. 61/982,753 filed Apr. 22, 2014, of the same title, which application is incorporated herein in its entirety by this reference.

### BACKGROUND

The present invention relates to hybrid systems and methods for acoustically or electronically monitoring chest sounds of a subject.

Acoustic stethoscopes have been in common usage since the early practice of medicine, and they are designed to transmit chest sounds such as heart and/or lung sounds from a subject to a listener. As mechanical devices, they have limitations including signal loss/attenuation, noise, and distortion. However, they are relatively inexpensive and durable and hence are very attractive to general practitioners and first responders in the field such as paramedics.

In recent times, electronic stethoscopes have emerged to overcome some of these limitations of acoustic stethoscopes. Most of these electronic stethoscopes are capable of amplifying and filtering the acoustic signals thereby substantially increasing their capabilities over the acoustic stethoscopes. However, these advantages come at a price, including increased complexity/costs, longer operator learning curve and reduced reliability.

It is therefore apparent that an urgent need exists for hybrid stethoscopes that are able to operate in either the acoustic mode or the electronic mode. These improved hybrid stethoscopes have the selectable capability of the electronic stethoscopes while substantially retaining the familiarity and reliability of the acoustic stethoscopes.

### SUMMARY

To achieve the foregoing and in accordance with the present invention, systems and methods for acoustically and/or electronically monitoring chest sounds is provided. In particular, hybrid acoustic electronic stethoscopes capable for the monitoring of patient heart and/or lung sounds with optional wireless transmission capability are provided.

In one exemplary embodiment, the hybrid acoustic and electronic stethoscope includes a chest piece having a diaphragm, an acoustic selector, a microphone, a signal processor and a speaker. The microphone is acoustically coupled to the diaphragm, and is configured to convert acoustic pressure signals, such as chest sounds, from the diaphragm into corresponding electronic signals. The speaker is acoustically coupled to the microphone via a signal processor, and the processor is configured to generate processed acoustic signals from the corresponding electronic signals.

In this embodiment, the acoustic selector having an acoustic input, e.g., an aperture in a hollow shaft, that can be selectably coupled to either the diaphragm or the speaker. A pair of binaurals is acoustically coupled to the output of the acoustic selector via an acoustic conduit. In turn, the binaurals provides the acoustic pressure signals or the processed acoustic signals to a user.

In some embodiments, the hybrid stethoscope has a wireless transmitter capable of sharing the processed acoustic signals with one or more other devices, e.g. in a training environment. These processed acoustic signals may also be displayed externally on a video monitor.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawings, in which:

FIG. 1 illustrates one embodiment of a hybrid acoustic and electronic stethoscope, in accordance with present invention;

FIG. 2 is a perspective view of exemplary chestpiece and selector for the hybrid stethoscope of FIG. 1;

FIGS. 3A and 3B are cross-sectional views of the chestpiece of FIG. 2, operating in acoustic mode and electronic mode, respectively;

FIG. 4 is a flow diagram illustrating the exemplary operation of the hybrid stethoscope of FIG. 1; and

FIG. 5 is a front-facing view of another embodiment of a hybrid stethoscope having a plurality of embedded ECG leads.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

Aspects, features and advantages of exemplary embodiments of the present invention will become better understood with regard to the following description in connection with the accompanying drawing(s). It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto. Hence, use of absolute and/or sequential terms, such as, for example, “will,” “will not,” “shall,” “shall not,” “must,” “must not,” “first,” “initially,” “next,” “subsequently,” “before,” “after,” “lastly,” and “finally,” are not meant to limit the scope of the present invention as the embodiments disclosed herein are merely exemplary.

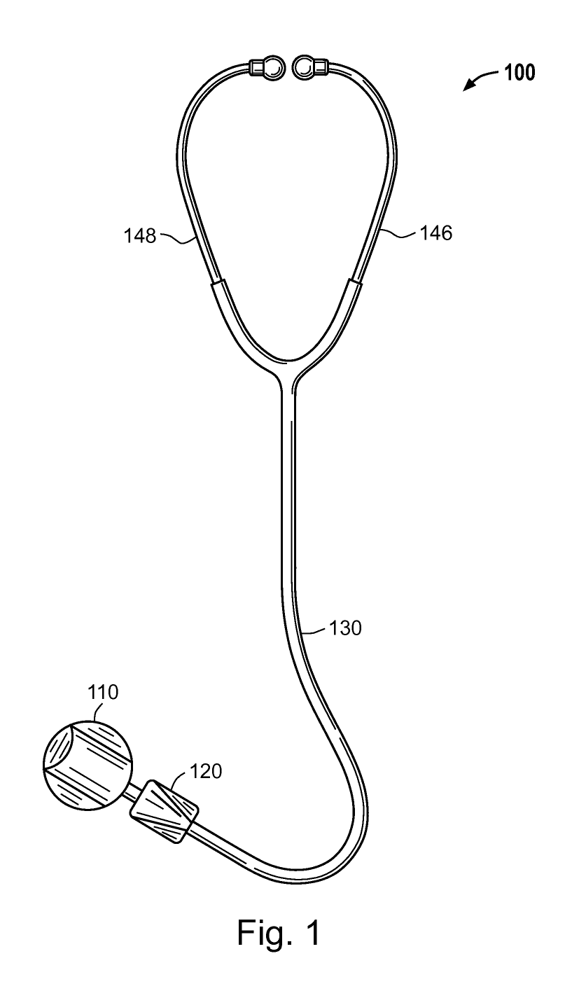

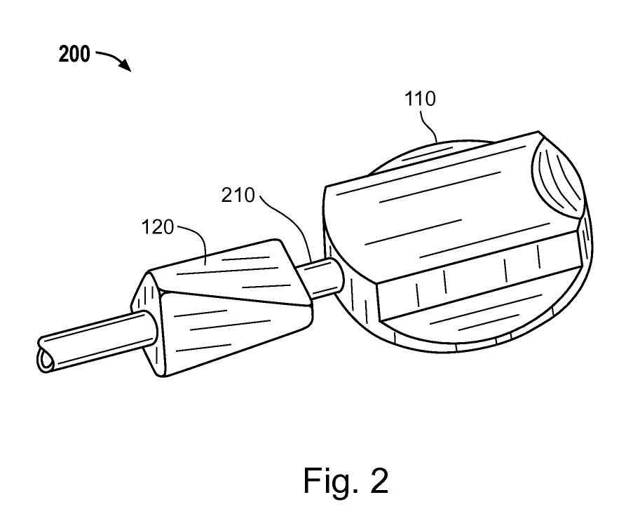

To facilitate discussion, FIG. 1 shows an exemplary embodiment of a hybrid acoustic and electronic stethoscope 100 having a chestpiece 110, a selector knob 120, an acoustic conduit 130 and a pair of binaurals 146, 148, acoustically coupled. FIG. 2 is a perspective view of chestpiece 110 and selector knob 120. In this embodiment, knob 120 is secured to a hollow external shaft 210 which is rotably coupled to chestpiece 110.

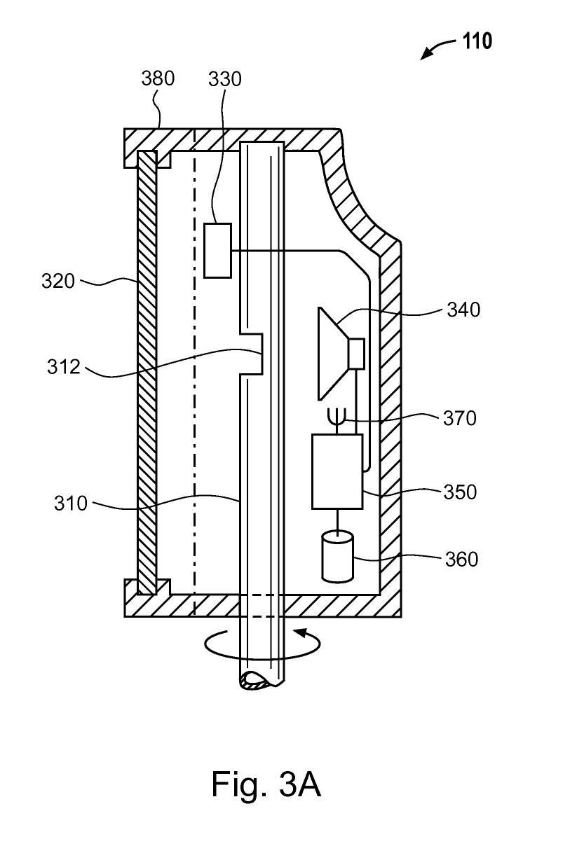

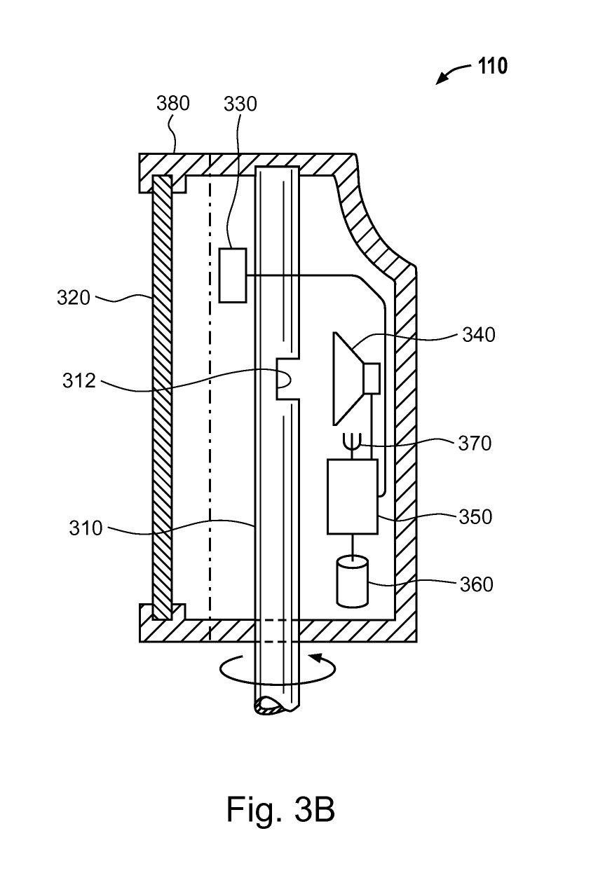

As shown in the cross-sectional cut-away views of FIGS. 3A and 3B, exemplary chestpiece 110 includes a hollow internal shaft 310, a diaphragm 320, a first electronic transducer 330, a second electronic transducer 340, a signal processor 350, a battery 360, an antenna 370 and an enclosure 380. Note that internal shaft 310 is secured to external shaft 210.

In this embodiment, transducer 330 is a microphone, transducer 340 is a speaker, and both transducers 330, 340 are coupled to signal processor 350. Signal processor 350 may also include or be coupled to an optional wireless transmitter (not shown) coupled to antenna 370, enabling chestpiece 110 to share information with other electronic device(s).

Depending on the user's needs, signal processor 350 can be powered by battery 360 which may be replaceable and/or rechargeable by using, for example, wireless inductive charging. Many other alternate and/or supplemental power sources are also possible, including solar cells and a/c adapters.

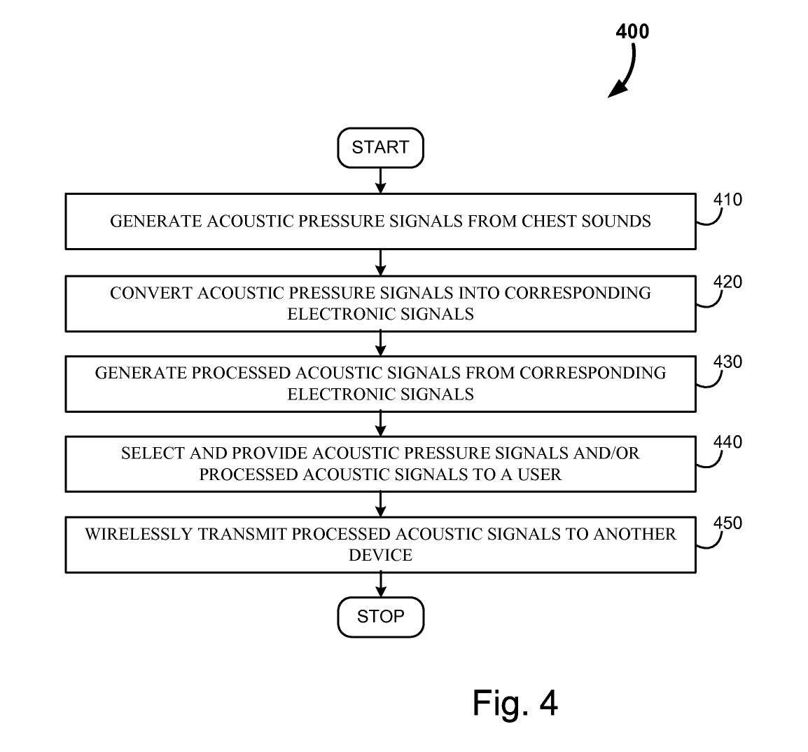

Referring to the flow diagram of FIG. 4 and FIG. 3A illustrating an acoustic mode of operation for chestpiece 110, hollow internal shaft 310 has an aperture 312 positioned to substantially face the diaphragm 320. In step 410, when a user, e.g., a physician or nurse, places the chestpiece 110 proximate to the chest wall of a subject, e.g., a patient, the diaphragm 320 picks up chest sounds such as heart and/or lung sounds of the subject, thereby generating acoustic pressure signals. Since aperture 312 is facing diaphragm 320, the acoustic pressure signals from diaphragm 320 are directed, via aperture 312 and acoustic conduit 130, towards the pair of binaurals 146, 148 (see step 440). In this acoustic mode of operation, the tactile feel/feedback and generated sounds of hybrid stethoscope 100 is very familiar to the typical user previously trained to use acoustic stethoscopes.

Referring again to the flow diagram of FIG. 4 and now to FIG. 3B illustrating an electronic mode of operation for chestpiece 110, the hollow shaft 310 has been rotated 180 degrees and aperture 312 is now repositioned to substantially face the second transducer 340, e.g., a speaker. Repositioning, i.e., rotating shaft 310, can be accomplished by, for example, rotating selector knob 120 with respect to chestpiece 110.

In step 410, when the user places the chestpiece 110 proximate to the chest wall of the subject, the diaphragm 320 picks up chest sounds of the subject, thereby generating acoustic pressure signals. The first transducer 330, e.g., a microphone, picks up these acoustic pressure signals and converts them into corresponding electronic signals for signal processor 350 (step 420). In step 430, signal processor 350 generates processed acoustic signals from the corresponding electronic signals via the second transducer 340. Since aperture 312 is now positioned to substantially face the second transducer 340, the processed acoustic signals from second transducer 340 are directed, via aperture 312 and acoustic conduit 130, towards the pair of binaurals 146, 148 (see step 440).

In this embodiment, the signal processor 350 is capable of amplifying and/or selectively filtering the corresponding electronic signals from the first transducer 330. For example, the user may wish to listen to chest sounds corresponding to the equivalent “diaphragm” or “bell” mode of an acoustic stethoscope.

As discussed above, the chestpiece 110 may be capable of wirelessly sharing processed acoustic signals with one or more other electronic devices such as televisions, tablets, smart phones or portable patient monitors. Wireless transmissions from chestpiece 110 can be based on standard protocols such as Bluetooth, Wi-Fi or public cellular phone network protocols, or based on proprietary protocols. It may also be possible for chestpiece 110 to display these processed acoustic signals on an external video monitor via a suitable communications port, e.g., by using a USB, VGA, S-Video, RCA/BNC (Composite video), DVI, HDMI, FireWire or Thunderbolt connector.

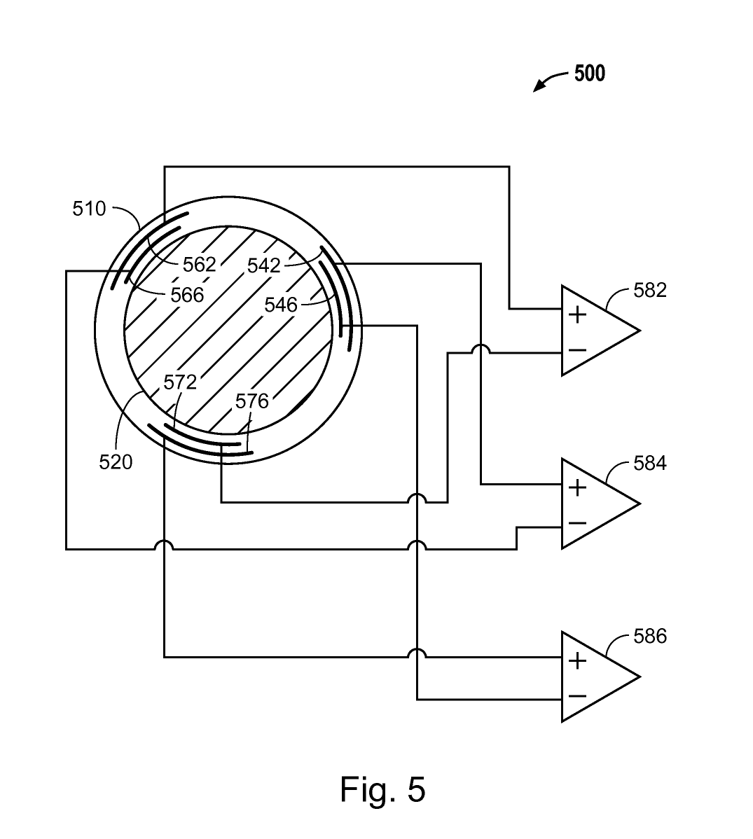

FIG. 5 illustrates the patient-facing front view of an alternate embodiment 500 of an exemplary enhanced chestpiece 510 having a diaphragm 520 and three pairs of electrical leads 542-546, 562-566 & 572-576 that may be coupled to corresponding trio of ECG sensors. Note that the corresponding trio of input op-amps 582, 584 & 586 (of the respective ECG sensors) and their respective input lead connectors are shown in traditional electronic circuitry drafting format for illustrative purposes only. In practice, these op-amps 582, 584 & 586 and their input lead connectors should ideally be contained and protected in a water resistant enclosure.

Many other modifications and additions are also possible. For example, the diaphragm may be part of an interchangeable assortment of diaphragm mode, bell mode, pediatric sized. It is also possible for a modified chestpiece, e.g. by modifying the hollow selector shaft, to provide three selectable modes: an acoustic only mode, an electronic only mode, or a combined acoustic and electronic mode. Miniaturized wrist-worn health status monitor with wireless capability are also possible.

In sum, the present invention provides hybrid acoustic electronic stethoscopes capable for the monitoring of patient heart and/or lung sounds with optional wireless transmission capability. The advantages of such hybrid stethoscopes include the ability to smoothly transition to acoustic-only mode when for example the electronics has failed or becomes unpowered.

While this invention has been described in terms of several embodiments, there are alterations, modifications, permutations, and substitute equivalents, which fall within the scope of this invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A hybrid acoustic and electronic stethoscope, the stethoscope comprising:
a chestpiece having a diaphragm configured to provide acoustic pressure signals;
a first electronic transducer acoustically coupled to the diaphragm, the first transducer configured to convert the acoustic pressure signals from the diaphragm into corresponding electronic signals;
a second electronic transducer coupled to the first transducer, the second transducer configured to process the corresponding electronic signals and to generate processed acoustic signals;
an acoustic selector having an input aperture configured to selectably couple to at least one of the diaphragm providing the acoustic pressure signals and the second transducer generating the processed acoustic signals; and
a pair of binaurals acoustically coupled to an output of the acoustic selector, the pair of binaurals configured to provide at least one of the acoustic pressure signals and the processed acoustic signals to a user.

2. The hybrid stethoscope of claim 1 wherein the pair of binaurals is acoustically coupled to the output of the acoustic selector via an acoustic conduit.

3. The hybrid stethoscope of claim 2 wherein the acoustic conduit is a flexible tube.

4. The hybrid stethoscope of claim 1 wherein the first transducer includes a microphone.

5. The hybrid stethoscope of claim 1 wherein the second transducer includes a speaker.

6. The hybrid stethoscope of claim 1 wherein the acoustic selector includes a hollow shaft with an aperture.

7. The hybrid stethoscope of claim 1 wherein the diaphragm, the first transducer and the second transducer are contained within an enclosure.

8. The hybrid stethoscope of claim 7 further comprising a battery coupled to the second transducer and wherein the battery is contained within the enclosure.

9. The hybrid stethoscope of claim 8 wherein the battery is rechargeable.

10. The hybrid stethoscope of claim 7 further comprising a wireless transmitter and an antenna coupled to the second transducer and wherein the transmitter and the antenna are contained within the enclosure.

11. The hybrid stethoscope of claim 10 wherein the wireless transmitter includes at least one of a Bluetooth transmitter, a Wi-Fi transmitter, and a cellular phone transmitter.

12. The hybrid stethoscope of claim 1 wherein the first transducer includes a signal processor configured to filter the acoustic pressure signals provided by the diaphragm.

13. The hybrid stethoscope of claim 12 wherein the filtering of the acoustic pressure signals includes at least one of a diaphragm mode and a bell mode.

14. The hybrid stethoscope of claim 1 wherein the second transducer includes a signal processor configured to filter the corresponding electronic signals provided by the first transducer.

15. The hybrid stethoscope of claim 14 wherein the filtering of the corresponding electronic signals includes at least one of a diaphragm mode and a bell mode.

16. A hybrid computerized method for monitoring heart and lung sounds of a subject, the method comprising:
generating acoustic pressure signals from at least one of a heart sound and a lung sound of a subject;
converting the acoustic pressure signals into corresponding electronic signals;
generating processed acoustic signals from the corresponding electronic signals;
selecting at least one of the acoustic pressure signals and the processed acoustic signals; and
providing the selected at least one of the acoustic pressure signals and the processed acoustic signals to a user.

17. The method of claim 16 further comprising wirelessly transmitting the processed acoustic signals to another device.

18. The method of claim 16 further comprising filtering at least one of the corresponding electronic signals and the processed acoustic signals.

19. The method of claim 18 wherein the filtering includes at least one of a diaphragm mode and a bell mode.
